(19) United States
(12) Patent Application Publication
(10) Pub. No.: US 2026/0124301 A1
(43) Pub. Date: May 7, 2026

(54) IMMUNE CELL ENGINEERED WITH IL-8 RECEPTOR AND USE THEREOF

(71) Applicant: Children's National Medical Center, Washington, DC (US)

(72) Inventors: Conrad Russell Y. Cruz, Washington, DC (US); Amy Hont, Washington, DC (US); Catherine Mary Bollard, Washington, DC (US)

(21) Appl. No.: 18/865,888

(22) PCT Filed: May 17, 2023
(86) PCT No.: PCT/US2023/022516

§ 371 Date: Nov. 14, 2024

Related U.S. Application Data
(60) Provisional application No. 63/343,215, filed on May 18, 2022.

(51) Int. Cl. A61K 40/11; A61K 35/17; A61K 40/13; A61K 40/15; A61K 40/42; C07K 16/24; C12N 5/0781; C12N 5/0783; C12N 5/10; C12N 15/867

(52) U.S. Cl. CPC A61K 40/11; A61K 35/17; A61K 40/13; A61K 40/15; A61K 40/424; A61K 40/4243; A61K 40/427; C07K 16/244; C12N 5/0635; C12N 5/0636; C12N 5/0646; C12N 5/10; C12N 15/867; A61K 2239/56; A61K 2239/57; C12N 2510/00

(57) ABSTRACT

The present invention provides a method for preparing an immune cell, such as a tumor-associated antigen-specific T cell (TAA-T cell), which is engineered with an IL-8 receptor. The present application also provides an immune cell, such as a T cell which is tumor-associated antigen-specific and is engineered with an IL-8 receptor. Immune cells (such as T cells) are genetically engineered to bind to IL-8 in the tumor environment that can serve as a potential strategy to improve immunotherapy for pediatric solid tumors.

### REFERENCE TO RELATED APPLICATION

This application claims priority to and the benefit of the filing date of U.S. Provisional Patent Application No. 63/343,215, filed on May 18, 2022, the entire contents of which are incorporated herein by reference.

### FIELD OF THE INVENTION

The present invention pertains to the fields of immune cells, such as B lymphocytes (B cells), natural killer cells (NK cells), or T cells, such as tumor-associated antigen-specific T cells (TAA-T cells).

### BACKGROUND OF THE INVENTION

The safety of tumor-associated antigen-specific T lymphocytes specific for WT1, PRAME, and Survivin against pediatric solid tumors, including Wilms tumor, were demonstrated previously in Phase I studies. The effects of attenuating interleukin 8 (IL-8) in the tumor environment on tumor and T cell function has not been previously explored.

### SUMMARY OF THE INVENTION

The present application provides a method for preparing an immune cell, such as a tumor-associated antigen-specific T cells (TAA-T cell), which is engineered with an Interleukin 8 (IL-8) receptor.

The present application also provides an immune cell, such as a tumor-associated antigen-specific T cell (TAA-T cell), which is engineered with an IL-8 receptor. Such immune cells, such as T cells, are genetically engineered to bind to IL-8 in the tumor environment that can serve as a potential strategy to improve immunotherapy for pediatric solid tumors.

In an embodiment, a TAA-T cell is transduced with an IL-8 receptor to bind, to serve as an IL-8 sink (to “sink IL-8”), or to respond to IL-8 which is known to be a chemokine released by tumors and immune cells in the tumor microenvironment.

The present application provides a mechanism to home immune cells, such as TAA-T cells, to areas of high IL-8 concentration and to sink IL-8 to decrease its effects. These immune cells, such as TAA-T cell products, can be infused into patients with high-risk malignancies to treat chemo-refractory diseases.

The invention described herein is further presented in the following numbered paragraphs:
1. An engineered immune cell engineered to express, on cell surface, an IL-8-binding protein capable of binding to IL-8.
2. The engineered immune cell of paragraph 1, wherein the IL-8-binding protein comprises:
   (1) a dominant negative IL-8 receptor (DNR) incapable of transducing IL-8 signaling upon IL-8 binding, or,
   (2) an antigen-binding portion of an antibody specific for IL-8.
3. The engineered immune cell of paragraph 2, wherein the IL-8-binding protein:
   (1) lacks intracellular signaling domain(s) of CXCR1/CXCR2, and/or,
   (2) transmits an activating signal for the engineered immune cell upon binding IL-8.
4. The engineered immune cell of any one of paragraphs 1-3, wherein the IL-8-binding protein is encoded by an expression construct (e.g., a vector) introduced into said immune cell.
5. The engineered immune cell of paragraph 4, wherein the expression construct/vector is a retroviral vector.
6. The engineered immune cell of any one of paragraphs 1-5, wherein the engineered immune cell is specific for WT1, PRAME, or Survivin.
7. The engineered immune cell of any one of paragraphs 1-6, wherein the engineered immune cell is a B cell, a natural killer (NK) cell, or a T cell.
8. The engineered immune cell of paragraph 7, wherein the T cell is a helper T cell, a cytotoxic T cell, a NK T cell, an iNK T cell, a gamma delta T cell, an alpha beta T cell, an antigen-specific T cell, a tumor-infiltrating lymphocyte (TIL), an engineered T cell receptor (TCR) cells, an engineered CAR-T cell, or an antigen-specific T cell.
9. The engineered immune cell of paragraph 8, wherein the antigen-specific T cell is a tumor-associated antigen-specific T (TAA-T) cell, a neoantigen specific T cell, or a virus specific T cell.
10. A method of treating cancer in a subject in need thereof, the method comprising administering to the subject a therapeutically effective amount of the immune cell of any one of paragraphs 1-9.
11. The method of paragraph 10, wherein the cancer is a pediatric solid tumor.
12. The method of paragraph 11, wherein the pediatric solid tumor is Wilms tumor, rhabdomyosarcoma, neuroblastoma, or melanoma.
13. The method of any one of paragraphs 10-12, wherein the cancer expresses or over-expresses IL-8.
14. The method of any one of paragraphs 10-13, wherein the cancer is a chemo-refractory cancer.
15. The method of any one of paragraphs 10-14, wherein the immune cell is specific for WT1, PRAME, or Survivin.
16. A method of producing the immune cell of any one of paragraphs 1-9, the method comprising introducing, into an isolated immune cell, an expression construct (e.g., vector) encoding an IL-8-binding protein capable of binding to IL-8, in order to enable expression of the IL-8-binding protein on the surface of the immune cell.
17. The method of paragraph 16, wherein the expression construct is a lentiviral or retroviral vector.
18. The method of paragraph 16 or 17, wherein the IL-8-binding protein comprises:
   (1) a dominant negative IL-8 receptor (DNR) incapable of transducing IL-8 signaling upon IL-8 binding, or,
   (2) an antigen-binding portion of an antibody specific for IL-8.
19. The method of any one of paragraphs 16-18, wherein the IL-8 binding protein:
   (1) lacks intracellular signaling domain(s) of CXCR1/CXCR2, and/or,
   (2) transmits an activating signal for the engineered immune cell upon binding IL-8.
20. The method of any one of paragraphs 16-19, wherein the immune cell is specific for WT1, PRAME, or Survivin.
21. The method of any one of paragraphs 16-20, wherein the engineered immune cell is a B cell, a natural killer (NK) cell, or a T cell.
22. The method of paragraph 21, wherein the T cell is a helper T cell, a cytotoxic T cell, a NK T cell, an iNK T cell, a gamma delta T cell, an alpha beta T cell, an antigen-specific T cell, a tumor-infiltrating lymphocyte (TIL), an engineered T cell receptor (TCR) cells, an engineered CAR-T cell, or an antigen-specific T cell.
23. The method of paragraph 22, wherein the antigen-specific T cell is a tumor-associated antigen-specific T (TAA-T) cell, a neoantigen specific T cell, or a virus specific T cell.

It should be understood that any one embodiment of the invention described herein, including embodiments described only in the examples or claims, can be combined with one or more additional embodiments of the invention, unless such combination is improper or expressly disclaimed.

### BRIEF DESCRIPTION OF THE DRAWING FIGURES

FIG. 1A shows IL-8 secretion of tumor cell lines according to an exemplary embodiment.

FIG. 1B shows REST trial patient IL-8 data according to an exemplary embodiment.

FIG. 2 shows the two native CXCR1 and CXCR2 IL-8 receptors, and four exemplary IL8 binding protein constructs of the invention according to an exemplary embodiment. Dominant-negative IL-8 receptors (IL8 DNR) bind IL-8 but lack intracellular signaling capability. These constructs were used to generate retroviral supernatant for transduction of T cells.

FIG. 3A shows the process for transducing T cells via retroviral transduction according to an exemplary embodiment. PBMCs were stimulated with anti-CD3/CD28 beads to generate T cells, then transduced with retrovirus containing plasmids for each IL8 binding protein constructs (IL8 DNRs).

FIG. 3B shows expression of the constructs according to an exemplary embodiment. Flow cytometry data demonstrates high levels expression of the IL-8 binding protein constructs on the surfaces of T cells compared to non-transduced T cells.

FIG. 4A shows migration of IL-8 DNR T cells toward media containing IL-8 according to an exemplary embodiment. IL8 DNR-1 and IL8 DNR-12 T cells show improved migration toward CTL media containing IL-8 compared to CTL media alone as measured by Transwell migration assay compared to non-transduced T cells.

FIG. 4B shows migration experiment according to an exemplary embodiment.

FIG. 5A shows ELISA sinking data according to an exemplary embodiment. IL8 DNR-2 expressing T cells were cultured in media containing soluble IL-8 for indicated time periods (1, 2, or 3 days), and remaining IL-8 in the media was measured by ELISA to evaluate IL-8 sinking at 3 different time points.

FIG. 5B shows ELISA sinking data according to an exemplary embodiment. IL8 DNR-2 transduced T cells demonstrated sinking of IL-8 when cultured in supernatant from RMS rhabdomyosarcoma tumor cells.

FIG. 6 shows that IL8 DNR-1, IL8 DNR-2, and IL8 DNR-12 transduced T cells show improved migration in media containing IL-8, compared to control (CTL) media alone (without IL-8), as measured by Transwell migration assay compared to non-transduced T cells (NT) at various time points according to an exemplary embodiment.

### DETAILED DESCRIPTION OF THE INVENTION

### 1. Overview

Elevations in serum IL-8 have been noted in patients with solid tumors, such as relapsed or refractory solid tumors that corresponded to disease progression, specifically in the context of T cell immunotherapy (Hont et al., J Clin Oncol. July 2019:JCO1900177). IL-8 makes up one component of the immunosuppressive tumor microenvironment (TME), and promotes invasion, metastasis, and angiogenesis among many effects (see David et al., Vaccines (Basel). June 2016; 4(3) doi:10.3390/vaccines4030022; Waugh and Wilson, Clin Cancer Res. Nov. 1 2008; 14(21): 6735-41; Xie, Cytokine Growth Factor Rev. December 2001; 12(4): 375-91; Schalper et al., Nat Med. 05 2020; 26(5): 688-692). IL-8 as a chemokine is involved in cell migration, allowing for influx of cells that contribute to an immune suppressive microenvironment. IL-8 elevation in patients (such as elevation detected through investigating cytokine expression in patient sera) can be potentially associated with disease progression.

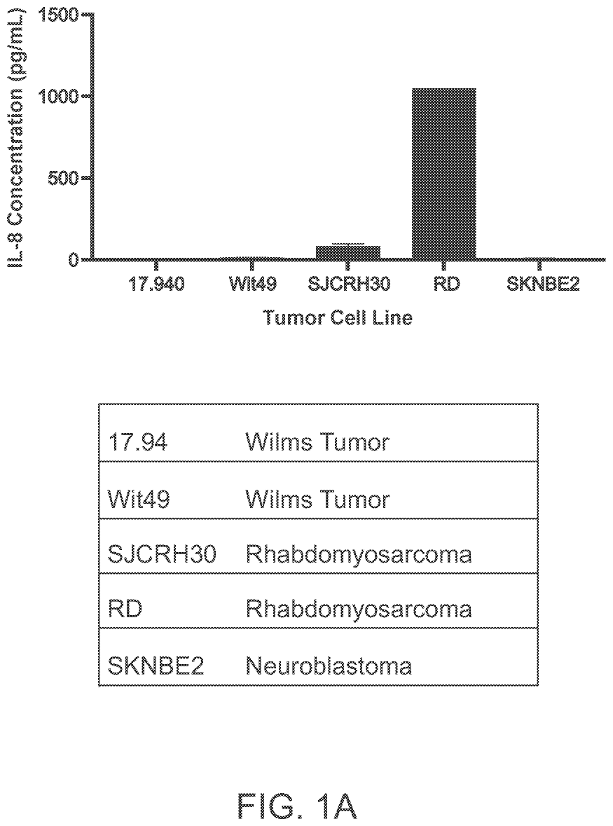

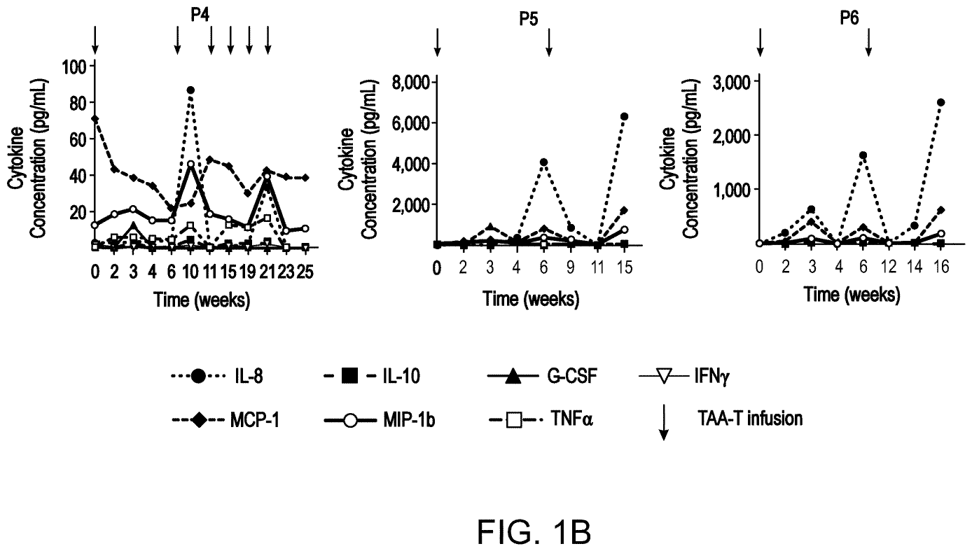

For example, FIG. 1A and FIG. 1B show elevated IL-8 in tumor cell supernatant and REST trial patient sera. In FIG. 1A, IL-8 concentration was measured by ELISA in supernatant collected from tumor cell lines. FIG. 1B similarly shows elevations in serum IL-8 which were measured by Luminex in serum collected from REST trial patients, which corresponded with disease progression.

The invention described herein demonstrates that immune cells, such as T cells, modified to express certain engineered receptors are capable of binding to and neutralizing IL8, such as those in the tumor microenvironment, and can increase susceptibility of tumors with IL-8 expression, such as pediatric solid tumors, to attack by such immune cells, such as tumor antigen-specific T cells (TAA-T cells).

Thus in one aspect, the invention provides an engineered immune cell engineered to express, on cell surface, an IL-8-binding protein capable of binding to IL-8.

As used herein, “immune cells” can include lymphocytes, such as B lymphocytes (B cells), natural killer (NK) cells, and T lymphocytes (T cells).

As used herein, T cells include naïve T cells, helper T cells (such as CD4⁺ T cells, or T_{H} cells, including T-helper1, T-helper2, T-helper17, Th9, Tfh, Th22, and regulatory T-cell), cytotoxic T cells (such as CD8⁺ T cells), memory T cells (including Central memory T cells (T_{CM} cells), Effector memory T cells (T_{EM} cells and T_{EMRA} cells), Tissue-resident memory T cells (T_{RM}), and Virtual memory T cells (T_{VM})), FOXP3⁺ T_{reg} cells, FOXP3⁻ Treg cells, innate-like T cells/unconventional T cells, NK T cells, mucosal associated invariant T (MAIT) cells, iNK T cells, gamma delta T cells, alpha beta T cells, antigen-specific T cells, tumor-infiltrating lymphocytes, engineered T cell receptor (TCR) cells, engineered CAR-T (chimeric antigen receptor (CAR) T) cells, and antigen-specific T cells which include tumor-associated antigen-specific T (TAA-T) cells, neoantigen specific T cells, or virus specific T cells.

The invention disclosed herein provide a novel therapeutic approach using such modified immune cells, such as T cells (e.g., TAA-T cells), since the safety of tumor-associated antigen-specific T lymphocytes specific for WT1, PRAME, and Survivin against pediatric solid tumors, including Wilms tumor, in Phase I studies were previously demonstrated.

Briefly, immune cells (such as T cells) genetically engineered to bind to IL-8 in the tumor environment are used as part of a potent strategy to improve immunotherapy for pediatric solid tumors. In certain embodiments, TAA-T cells are transduced with an IL-8 binding moiety, such as a dominant negative IL-8 receptor (IL8 DNR), in order to bind, “sink,” or respond to IL-8 which is known to be a chemokine released by tumors. IL-8 can attract such modified immune cells (such as T cells) to the tumor environment and potentially support tumor growth by encouraging metastases or immune suppression.

Thus, in certain embodiments, in the engineered immune cell of the invention, the IL-8-binding protein comprises: (1) a dominant negative IL-8 receptor (DNR) incapable of transducing IL-8 signaling upon IL-8 binding, or, (2) an antigen-binding portion of an antibody specific for IL-8. For example, the dominant negative IL-8 receptor may (1) lack intracellular signaling domain(s) of CXCR1/CXCR2, and/or, (2) transmits an activating signal for the engineered immune cell (such as T cell, e.g., TAA-T cell) upon binding IL-8.

In other words, the present application provides a mechanism to guide or “home” immune cells, such as TAA-T cells, to areas of high IL-8 concentration, such as the tumor microenvironment, and sequester or “sink” IL-8 to decrease its effects, including chemoresistance and metastasis. Further, the immune cells, such as TAA-T cells, may be further stimulated or activated upon binding to IL-8 through the engineered IL-8-binding protein. These engineered immune cell products, such as engineered TAA-T cell products, could be infused into patients with high-risk malignancies to treat chemo-refractory disease.

In certain embodiments, immune cells (such as T cells) are engineered to stably express IL-8 dominant negative receptors, and these receptors were able to sink IL-8 signaling and improve immune cell (such as T cell) migration. Immune cells (such as T cells) modified to express receptors are capable of binding and neutralizing IL8 to increase susceptibility of pediatric solid tumors to attack by such immune cells (such as TAA-T cells).

In an illustrative embodiment, as shown in the examples below, at least four illustrative IL-8 neutralizing receptor construct producer cell lines were manufactured and used to transduce T cells expanded using anti-CD3/28 Dyna beads. IL-8 receptor surface expression was measured by flow cytometry and compared to surface expression of the receptor backbone alone, and initial experiments to test their functions (migration) were performed.

In certain embodiments, immune cells (such as T cells) are engineered to stably express an IL-8-binding protein that comprises an antigen-binding portion of an antibody specific for IL-8. The antigen-binding portion can be any known in the art, including but not limited to Fab, Fab′, F(ab′)₂, Fd, single chain Fv or scFv, disulfide linked Fv, V-NAR domain, IgNar, intrabody, IgGΔCH2, minibody, F(ab′)₃, tetrabody, triabody, diabody, single-domain antibody, DVD-Ig, Fcab, mAb2, (scFv)₂, or scFv-Fc. In certain embodiments, the scFv component of the antigen-binding portion has a VH domain N-terminal to the VL domain, or vice versa. Optionally, the VH and VL domains are linked by a flexible linker of appropriate length. The flexible linker can be any of those known in the art, including linkers with 0-20 repeats each comprising of Gly and Ser residues, such as GS repeats, G₂S repeats, G₃S repeats, G₄S repeats, etc.

The dominant negative IL-8 receptor and the antigen-binding portion of an antibody specific for IL-8 are incapable of transmitting an IL-8 signal inside the engineered immune cell expressing such IL-8 binding protein on the surface.

IL-8 activates pertussis toxin-sensitive and receptor-coupled G proteins including Gα14, Gα16, Gαi2 and Gαi3. These heterotrimeric G proteins, upon dissociation and conversion of the alpha subunit to the GTP-bound active form, activate phospholipase C β2 (PLCβ2), RhoA and phosphatidylinositol 3-kinase (PI3K). IL-8 also stimulates phosphatidylinositol 4-phosphate kinase (PIP-K), which synthesizes phosphatidylinositol 4,5-biphosphate (PIP2), the source of IP3.

Thus, in certain embodiments, the dominant negative IL-8 receptor contains mutations in the cytoplasmic portion of the wt IL-8 receptor that substantially reduces or eliminates IL-8 signaling. Such mutations may include point mutations and/or deletion that prevents the activation of the downstream heterotrimetic G proteins to prevent their activation. The deletion may be deletion of the intracellular signaling domains of the wt IL-8 receptor.

In certain embodiments, the dominant negative IL-8 receptor or the antigen-binding portion of an antibody specific for IL-8 transmits an activating signal for the engineered immune cell upon binding IL-8. This is not unlike CAR T cells having a chimeric antigen receptor (CAR) that comprises an extracellular binding domain for a ligand, such as an antibody or antigen-binding portion thereof that specifically binds a target protein (such as an antigen), and upon binding such target, transmits an activation signal through the transmembrane domain to the cytoplasmic tail that may comprise signaling function, such as one or more ITAM domains.

Thus, in certain embodiments, the IL-8-binding protein of the invention may comprises a signaling domain that transmits an activating signal for the engineered immune cell upon binding IL-8. In certain embodiments, the immune cell is a T cell, and the IL-8 binding protein comprises a cytoplasmic/intracellular T cell signaling domain. In certain embodiments, the T cell signaling domain comprises an immunoreceptor tyrosine-based activation motif (ITAM) present in the cytoplasmic domain of CD3-zeta. In certain embodiments, the T cell signaling domain comprises one or more chimeric domains from T cell co-stimulatory proteins, such as that from CD28, CD27, CD134 (OX40), and/or CD137 (4-1BB). In certain embodiments, the T cell signaling domain comprises a CD3-zeta cytoplasmic domain. In certain embodiments, the T cell signaling domain comprises a CD3-zeta cytoplasmic domain and a co-stimulatory domain, such as that from CD28, CD27, CD134 (OX40), and/or CD137 (4-1BB). In certain embodiments, the T cell signaling domain comprises a CD3-zeta cytoplasmic domain and multiple co-stimulatory domains, such as CD28-41BB or CD28-OX40.

In certain embodiments, the immune cell is a NK cell, and the IL-8 binding protein comprises a cytoplasmic/intracellular NK cell signaling domain that activates NK cells. In certain embodiments, the NK cell signaling domain transmits an activating signal of one or more of CD94, CD28, NKG2D, CD40L, IL-12R, 2B4, KIR-S, IL-15Ra, or a cytokine receptor (such as IL-1, -2, -12, -18, -21, TNFa, or Type I IFN).

In certain embodiments, the immune cell is a B cell, and the IL-8 binding protein comprises a cytoplasmic/intracellular B cell signaling domain that activates B cells. In certain embodiments, the B cell signaling domain transmits an activating signal of CD40.

Any IL-8 receptor can be used in the present invention (e.g., for generating the dominant negative IL-8 receptor). In certain embodiments, the IL-8 receptor is a human IL-8 receptor, such as IL-8RA (or Human CXCR1, see GenBank: AAA64378.1, incorporated herein by reference). In certain embodiments, the IL-8 receptor is human IL-8RB (or Human CXCR2, see GenBank: AAA36108.1, incorporated herein by reference). In certain embodiments, the IL-8 receptor is a rabbit IL-8RA (see SwissProt Accession #P21109, incorporated herein by reference). In certain embodiments, the IL-8 receptor is a Gorilla IL-8RA (see SwissProt Accession #P55919, incorporated herein by reference). In certain embodiments, the IL-8 receptor is a Norwegian rat IL-8RA (see SwissProt Accession #P70612, incorporated herein by reference). In certain embodiments, the IL-8 receptor is a Chimpanzee IL-8RA (see SwissProt Accession #P55920, incorporated herein by reference). In certain embodiments, the IL-8 receptor is a mammalian IL-8RA or IL-8RB that is at least about 80%, 85%, 90%, 95%, 97%, 99% identical to human IL-8RA or IL-8RB, which sequences can be readily retrieved from public database such as NCBI, GenBank, SwissProt, JPD, EMBL etc using human IL-8RA or IL-8RB described above as query sequence.

In certain embodiments, the IL-8-binding protein is encoded by an expression construct (e.g., a vector) introduced into the immune cell. The expression construct can be any vector suitable for expression in mammalian cells, such as plasmids, cosmids, bacmids, YACs, BACs, and viral vectors, including retroviral vectors, adenoviral vectors, adeno associated viral vectors (AAVs), lentiviral vectors, etc.

In certain embodiments, the expression construct is a retroviral vector. For example, in certain embodiments, the IL-8 neutralizing receptor (IL-8 binding protein) transgenes are designed and transfected into retroviral producer cell lines to generate retroviral vectors capable of infecting mammalian immune cells, such as T cells.

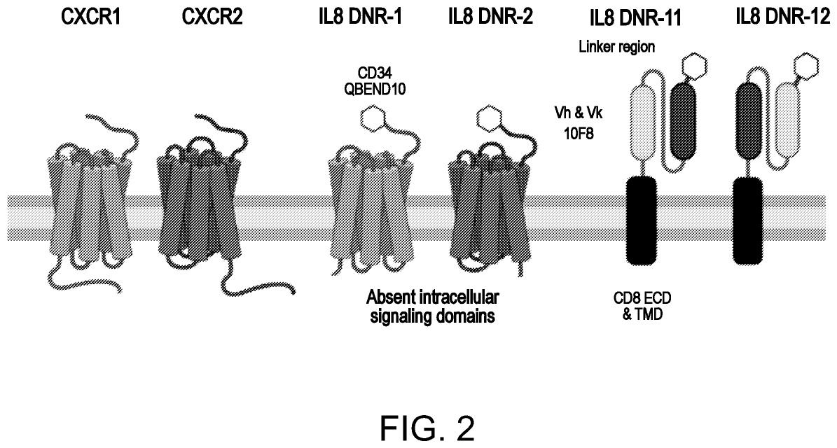

Retrovirus from producer cell lines containing the subject IL-8 binding protein (e.g., the IL8 neutralizing receptor, such as IL8 DNR) transgenes can be used to transduce immune cells (such as T cells), and induce surface expression of such IL-8 binding proteins. In certain embodiments, the surface expression can be facilitated by using flow cytometry for a tag fused to the IL-8 binding protein, such as the CD34 QBEND10 tag (see FIG. 2).

Retroviral packaging of transgenes is well known in the art. See, for example, WO1989007150A1, U.S. Pat. No. 5,591,624A, WO1993003743A1, U.S. Pat. No. 5,747,307A, WO1994003622A1, WO1994009120A1, WO1997007225A2, WO1997021825A1, etc., any of which can be used or adapted for use in the methods of the invention. All references are incorporated herein by reference.

In certain embodiments, the engineered immune cell is specific for WT1 (Wilms Tumor 1), PRAME (PReferentially expressed Antigen in MElanoma), or Survivin.

Wilms tumor protein (WT33) is a protein encoded by the WT1 gene in human. It encodes a transcription factor that contains four zinc finger motifs at the C-terminus and a proline/glutamine-rich DNA-binding domain at the N-terminus. It has an essential role in the normal development of the urogenital system, and it is mutated in a subset of patients with Wilms' tumor, the gene's namesake. Mutations of Wilms' tumor suppressor gene1 (WT1) are associated with embryonic malignancy of the kidney, Wilms tumor, and Denys-Drash syndrome (DDS), leading to nephropathy and genital abnormalities. WT1 protein has been found in the cell nuclei of 75% of mesotheliomas and in 93% of ovarian serous carcinomas, as well as in benign mesothelium and fallopian tube epithelium. A vaccine that induces an acquired immune response against WT1 is in clinical trials for various cancers. T cell therapies (TCR-T) are also being tested in clinical trials for leukemia.

PRAME (preferentially expressed antigen of melanoma) is a protein that in humans is encoded by the PRAME gene. This gene encodes an antigen that is predominantly expressed in human melanomas and that is recognized by cytolytic T lymphocytes. It is not expressed in normal tissues, except testis. The overexpression of PRAME in tumor tissues (such as acute leukemias) and relative low levels in normal somatic tissues make it an attractive target for cancer therapy.

Survivin, also called baculoviral inhibitor of apoptosis repeat-containing 5 or BIRC5, is encoded by the BIRC5 gene in human. It is a member of the inhibitor of apoptosis (IAP) family, and functions to inhibit caspase activation, thereby leading to negative regulation of apoptosis or programmed cell death. Disruption of survivin induction pathways leads to increase in apoptosis and decrease in tumour growth. The survivin protein is expressed highly in most human tumors and fetal tissue, but is completely absent in terminally differentiated cells, and thus presents an attractive target for cancer therapy that would discriminate between transformed and normal cells. Survivin expression is also highly regulated by the cell cycle and is only expressed in the G2-M phase. It is known that Survivin localizes to the mitotic spindle by interaction with tubulin during mitosis and may play a contributing role in regulating mitosis.

In certain embodiments, the engineered immune cell is a B cell, a natural killer (NK) cell, or a T cell. In certain embodiments, the T cell is a helper T cell, a cytotoxic T cell, a NK T cell, an iNK T cell, a gamma delta T cell, an alpha beta T cell, an antigen-specific T cell, a tumor-infiltrating lymphocyte (TIL), an engineered T cell receptor (TCR) cells, an engineered CAR-T cell, or an antigen-specific T cell. In certain embodiments, the antigen-specific T cell is a tumor-associated antigen-specific T (TAA-T) cell, a neoantigen specific T cell, or a virus specific T cell.

In further embodiments, similarly designed binding proteins for other cytokines can serve as sinks for other cytokines, such as IL10, IL6, etc. The subject immune cells, including T cells, can be similarly engineered to express binding proteins for IL-6 and/or IL-10.

Another aspect of the invention provides a method of treating cancer in a subject in need thereof, the method comprising administering to the subject a therapeutically effective amount of the immune cell (such as T cell) of the invention described herein.

In certain embodiments, the cancer is a pediatric solid tumor.

In certain embodiments, the pediatric solid tumor is Wilms tumor, rhabdomyosarcoma, neuroblastoma, or melanoma.

In certain embodiments, the cancer expresses or over-expresses IL-8.

In certain embodiments, the cancer is a chemo-refractory cancer.

In certain embodiments, the immune cell is specific for WT1, PRAME, or Survivin.

Another aspect of the invention provides a method of producing the immune cell of the invention, the method comprising introducing, into an isolated immune cell, an expression construct (e.g., vector) encoding an IL-8-binding protein capable of binding to IL-8, in order to enable expression of the IL-8-binding protein on the surface of the immune cell.

In certain embodiments, the expression construct is a lentiviral vector or retroviral vector.

In certain embodiments, the IL-8-binding protein comprises: (1) a dominant negative IL-8 receptor (DNR) incapable of transducing IL-8 signaling upon IL-8 binding, or, (2) an antigen-binding portion of an antibody specific for IL-8.

In certain embodiments, the IL-8 binding protein: (1) lacks intracellular signaling domain(s) of CXCR1/CXCR2, and/or, (2) transmits an activating signal for the engineered immune cell upon binding IL-8.

In certain embodiments, the immune cell is specific for WT1, PRAME, or Survivin.

In certain embodiments, the engineered immune cell is a B cell, a natural killer (NK) cell, or a T cell.

In certain embodiments, the T cell is a helper T cell, a cytotoxic T cell, a NK T cell, an iNK T cell, a gamma delta T cell, an alpha beta T cell, an antigen-specific T cell, a tumor-infiltrating lymphocyte (TIL), an engineered T cell receptor (TCR) cells, an engineered CAR-T cell, or an antigen-specific T cell.

In certain embodiments, the antigen-specific T cell is a tumor-associated antigen-specific T (TAA-T) cell, a neoantigen specific T cell, or a virus specific T cell.

Data presented herein demonstrates that IL8 DNR immune cells (such as T cells) demonstrate “sinking” of IL-8 in vitro at varying concentrations of IL-8, and preliminary data suggest sinking of tumor supernatant containing endogenously produced IL-8. Migration of IL8 DNR transduced immune cells such as T cells can be improved compared to non-transduced immune cells (T cells), such as improved migration in the presence of soluble IL-8. Such IL8 DNR immune cells (such as T cells) play a role in vivo to improve immune cell/TAA-T efficacy by both sinking IL-8 signaling and improving migration toward an endogenous IL-8 gradient for immune cells/T cells. Overall, the invention described herein provides a novel approach to overcome the immunosuppressive TME by abrogating IL-8 signaling, and thus improves immunotherapy and leads to better outcomes in cancer treatment, especially in pediatric patients with solid tumors.

Specifically, the invention described herein further enhances the anti-tumor effects of immune cells, such as multi-tumor associated antigen-specific T cells (TAA-T cells), by expression of an IL-8 binding protein that acts like a dominant negative IL-8 receptor, in order to target/home such immune cells (such as TAA-T cells) to the TME enriched with endogenously expressed IL-8 at the local TME, and once there, to kill tumor cells as well as to sequester the IL-8 in the TME.

Thus far, preliminary results have shown that T cells transduced with IL-8 binding proteins were able to serve as IL-8 sink and sequester IL-8 from the environment, and such T cells have improved migration in vitro to areas of high IL-8 concentration. The data supports the ability of such TAA-T cells transduced to express IL-8 binding proteins to neutralize IL-8 in the TME, to enhance T cell trafficking, and to improve the anti-tumor effects of such TAA-T cells in vivo.

The subject IL-8 binding protein-expressing immune cells (such as T cells) can be used in cell therapy for the treatment of a wide range of malignancies and immunologic conditions.

All such immune cells products, including antigen-specific T cell products, such as TAA-T cells products, and chimeric antigen receptor (CAR) T cell products, can be isolated/produced/generated using any art-recognized methods, by incorporating the subject IL-8 binding moieties, in order to mitigate the immunosuppressive tumor microenvironment.

Other immune cell therapy products, including but not limited to natural killer (NK) cells, NK T cells, gdT cells, iNKT cells, B cells, tumor-infiltrating lymphocytes (TILs), or engineered T cell receptor (TCR) cells, can similarly be manufactured with the subject IL-8 binding proteins.

In certain embodiments, the subject immune cells, such as non-specific T cells engineered to express on their surface the subject IL-8 binding proteins can be used in combination with other forms of immunotherapy, including but not limited to immune checkpoint inhibitors, bi-specific T cell engagers (BiTEs), bi- and tri-specific killer engagers (BiKEs, TriKEs), or oncolytic viruses to enhance clinical response.

In certain embodiments, the checkpoint inhibitor is a CTLA4 inhibitor, a PD-1 inhibitor, or a PD-L1 inhibitor. In certain embodiments, the checkpoint inhibitor is an antibody or antigen-binding portion thereof. In certain embodiments, the checkpoint inhibitor is a small molecule checkpoint inhibitor (such as AUNP12, CA-170 or BMS-986189).

In certain embodiments, the PD-1 inhibitor is an antibody, such as nivolumab, Pembrolizumab, Cemiplimab, Dostarlimab, Retifanlimab, Spartalizumab, Vopratelimab, Sintilimab, Tislelizumab, Toripalimab, INCMGA00012, AMP-224, AMP-514, or Acrixolimab. In certain embodiments, the PD-L1 inhibitor is an antibody, such as Atezolizumab, Avelumab, Durvalumab, KN035, or Cosibelimab (CK-301).

In certain embodiments, the CTLA4 inhibitor is an antibody, such as Ipilimumab.

In addition to its applications for cell therapy, these immune cell (T cell) products can also be part of targeted gene therapy approaches that specifically modify certain cells in vivo.

Throughout this description, the preferred embodiments and examples provided herein should be considered as exemplar, rather than as limitations of the present application.

Further features of the inventive concept, its nature and various advantages will be more apparent from the following examples, taken in conjunction with the accompanying figures.

### EXAMPLES

The features and properties of the present application are shown in the examples which illustrate the benefits and advantages of the present invention.

### Example 1 Generation of IL-8 Binding Proteins

This example illustrates the construction of different types of IL-8 binding proteins for use in the subject TAA-T cells.

One type of IL-8 binding protein is dominant negative version of the natural IL-8 receptor (DNR), which substantially retains binding affinity of the natural IL-8 receptors to IL-8, but substantially lacks ability to transduce intracellular signaling upon binding to IL-8.

Two types of human IL-8 receptors, type A and B, have been identified. The Type A (IL-8RA, also known as CMKAR1, CXCR1, CKR-1, CDw128a, or CD181) and Type B (IL-8RB, also known as CXCR2, CKR-2, or CDw128b). See FIG. 2. The IL-8 receptors are 7-transmembrane proteins, in that they contain 7 alpha helices that each span the phospholipid bilayer of a cell membrane. IL-8RA is a peptide of 350 amino acids, and IL-8RB is composed of 355 amino acids. Receptors A and B share 78% of their sequence identity, and are considered to be the only two biologically significant receptors of IL-8. However, Duffy antigen on erythrocytes can also bind IL-8 (and a number of other cytokines/chemokines). Cells transfected with type A IL-8 receptor cDNA bind only IL-8 with a high affinity, whereas cells transfected with type B bind neutrophil-activating protein 2 (NAP-2) and other small receptor molecules of the CXC chemokine family, as well as IL-8, with similar high affinity.

The translated proteins are approximately 40 kD, about 20 kD less than the native purified receptors from the surface of neutrophils. This difference could be due to the N-terminus glycosylation that occur post-translation and contribute to an increase in apparent size of the mature receptor.

The amino terminus of the receptor is located on the extracellular side of the protein, and functions to determine the binding specificity of ligands (IL-8) to the receptors. Meanwhile, the carboxyl terminus of the receptor is located on the intracellular side of the protein, and is rich in serine and threonine residues (a characteristic of many proteins of the 7-transmembrane G-protein coupled receptor family). The C-terminus is a target for phosphorylation and exhibits kinase activity. This is the beginning of IL-8 signaling pathways and phosphorylation cascades to a series of downstream signaling events that culminating in recruitment of neutrophils and angiogenesis, the development and growth of new blood vessels.

Thus in some embodiments, a DNR of IL-8 can be constructed by substantial complete deletion of the intracellular domain of the natural receptor CXCR1 or CXCR2. Constructs 1 (IL8 DNR-1 in FIGS. 2) and 2 (IL8 DNR-2 in FIG. 2) used in the examples below were created based on CXCR1 and CXCR2, respectively.

In another embodiment, a DNR of IL-8 can be constructed by substituting the Ser or Thr residues (by, for example, Ala) as targets for intracellular phosphorylation.

In another embodiment, a DNR of IL-8 can be constructed by mutating the key residues in the catalytic center of the intracellular kinase domain of the CXCR1/CXCR2.

Another type of IL-8 binding protein is antibody or antigen-binding fragment or portion thereof (such as scFv) specific for IL-8. For example, Constructs 11 (IL8 DNR-11 in FIGS. 2) and 12 (IL8 DNR-12 in FIG. 2) used in the following experiments contain the variable domains (VH/VL) from both the heavy and light chain of antibody 10F8, an antibody specific for human IL-8, attached with a linker domain to form an scFv (see FIG. 2). This IL-8 binding region connects to a transmembrane (TMD) and extracellular domain (ECD) from CD8, to allow for surface expression of these two constructs. The VH can be N- or C-terminal to the VL in the scFv. See IL8 DNR-11 and -12, respectively, in FIG. 2.

Not required for IL-8 binding, but useful to track or demonstrate surface expression of the IL-8 binding proteins, is a CD34 QBEND10 epitope inserted into the extracellular portion of Constructs 1-4. See the hexigon shaped moieties in FIG. 2. This epitope serves as a marker for cell surface expression, and can be used to distinguish wild-type CXCR1 or CXCR2 expression on T cells.

### Example 2 Creating IL-8 Binding Protein Construct Producer Cell Lines

The coding sequences for these IL-8 binding proteins described in Example 1 were introduced/transfected into retroviral producer cell lines to produce retroviral vectors for transducing TAA-T cells. Four retroviral cell lines expressing four different IL-8 binding protein transgenes were generated.

Specifically, 293T/Phoenix Eco cells were transfected with plasmids encoding the various IL-8 binding protein constructs (Constructs 1-4). 293T/Phoenix Eco is a second-generation retrovirus producer cell line for the generation of helper-free ecotropic (for infecting mouse/rat cells) and also amphotropic (for infecting mammalian cells) retroviruses. The lines are based on the 293T cell line—a human embryonic kidney line transformed with adenovirus E1a and carrying a temperature sensitive T antigen co-selected with neomycin. The unique feature of this cell line is that it is highly transfectable with either calcium phosphate mediated transfection or lipid-based transfection protocols. The lines were created by placing into 293T cells constructs capable of producing gag-pol, and envelope protein for ecotropic and amphotropic viruses.

The supernatant was then used to transduce PG13 cells. PG13 cells were derived from TK-NIH/3T3 cells. It is a retroviral packaging cell line based on the gibbon ape Leukaemia Virus (GaLV). Introduction of retroviral vectors results in the production of retrovirus virions capable of infecting cells from many species excluding mice. Transduced PG13 cells were sorted and cultured, and the PG13 supernatant was used for transduction of TAA-T cells.

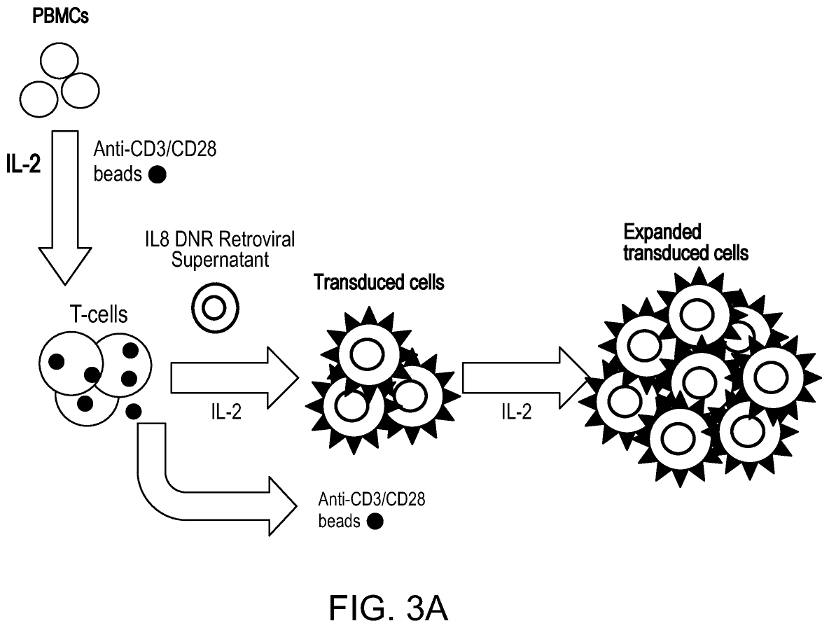

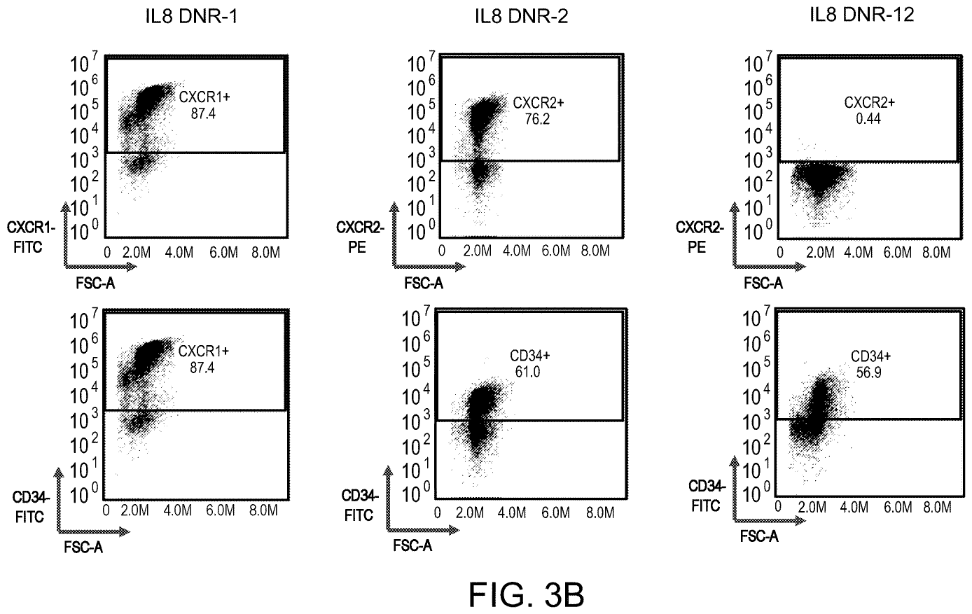

Transduction experiments were performed on T cells expanded using anti CD3/28 Dynabeads (see FIG. 3A). Specifically, T cells were isolated from peripheral blood mononuclear cells (PBMCs) stimulated with anti-CD3/CD28 Dynabeads, and cultured in media containing IL-2. The T cells were then transduced with retroviral supernatant from the retroviral producer cell lines. IL-8 binding protein surface expression was measured by flow cytometry for CD34 QBEND10, and was compared to surface expression of the receptor backbone alone. All experiments with transduced products were compared to non-transduced controls.

The data showed that these transduced T cells demonstrated high levels of surface expression of the IL8 binding protein Constructs 1-4 described in Example 1, as measured by flow cytometry staining for the CD34 QBEND10 epitope marker. Durable expression had also been demonstrated on cells following freeze-thaw cycles, demonstrating the stability of these constructs and continued surface expression (FIG. 3B).

### Example 3 Transduced T Cells Serve as IL-8 Sink Compared to Non-Transduced T Cells

This experiment demonstrates that the subject TAA-T cells expressing surface IL-8 binding protein can serve as IL-8 sink to sequester IL-8 in the tumor microenvironment (TME), thus reducing the available IL-8 in the TME.

Specifically, T cells transduced with various IL-8 binding protein constructs (see Example 2) were cultured in the presence of media containing soluble IL-8 at predetermined concentrations. IL-8 neutralization is quantified using Enzyme-Linked Immunosorbent Assay (ELISA) specific for IL-8. Transduced T cells were compared to non-transduced T cells alone as control.

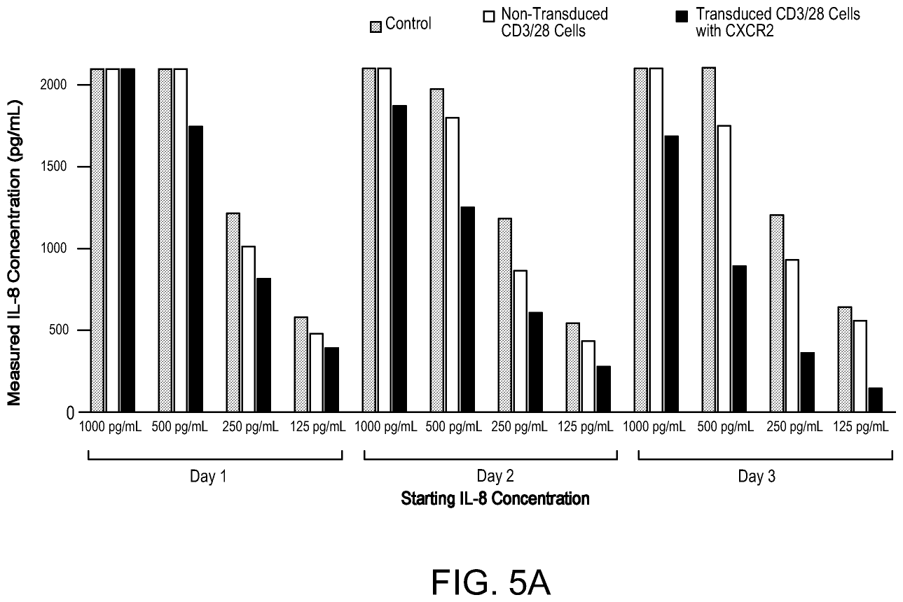

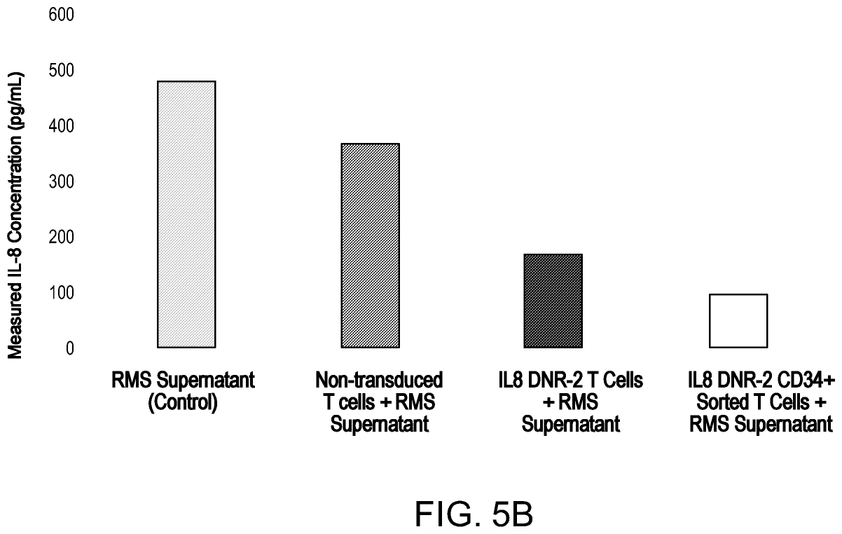

Transduced T cells expressing surface IL-8 binding protein demonstrated their ability as IL-8 “sink” in vitro at varying concentrations of IL-8. Preliminary data also suggested sequestration of endogenously produced IL-8 in the tumor microenvironment. Specifically, cultures of both IL8 DNR-2-transduced and non-transduced T cells in media containing exogenous IL-8 at concentrations ranging from 125 pg/mL to 1,000 pg/mL were performed, and remaining IL-8 concentration was measured in the supernatant by IL-8 ELISA at days 1, 2, and 3. Results demonstrated that IL8 DNR-2 transduced T cells were able to reduce IL-8 levels in culture compared to non-transduced T cells, suggesting that IL8 DNR T cells served as IL-8 sink in vitro (See FIGS. 5A and 5B).

### Example 4 Transduced T Cells Improved Migration to Areas of High IL-8 Concentration

Migration assays assess the role of IL-8 signaling in cellular homing using an in vitro transwell assay model. Specifically, transwell migration assays were utilized to assess the migration capabilities of IL-8 binding protein-transduced T cells compared to non-transduced T cells, in media containing soluble IL-8 at predetermined concentrations. Controls included media without IL-8 and supernatant from tumors known to secrete IL-8.

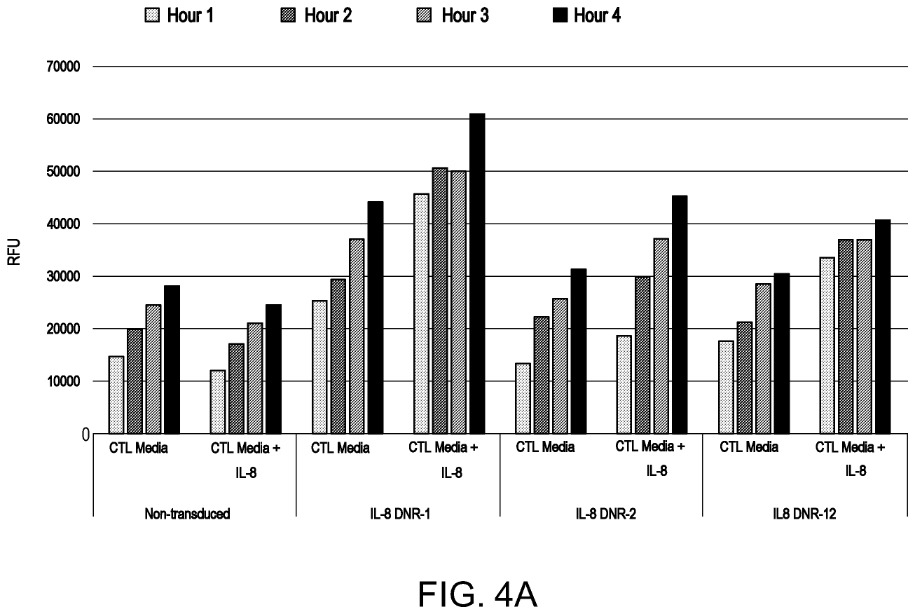

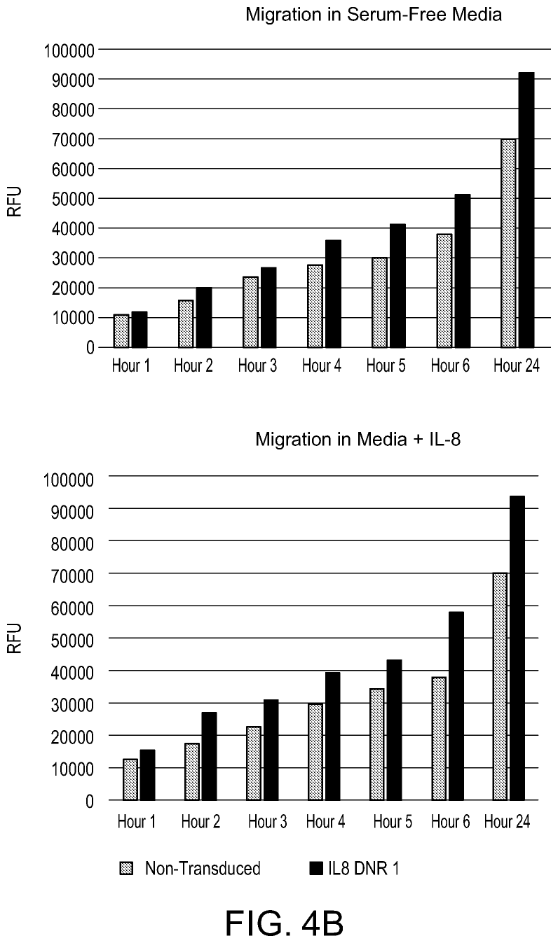

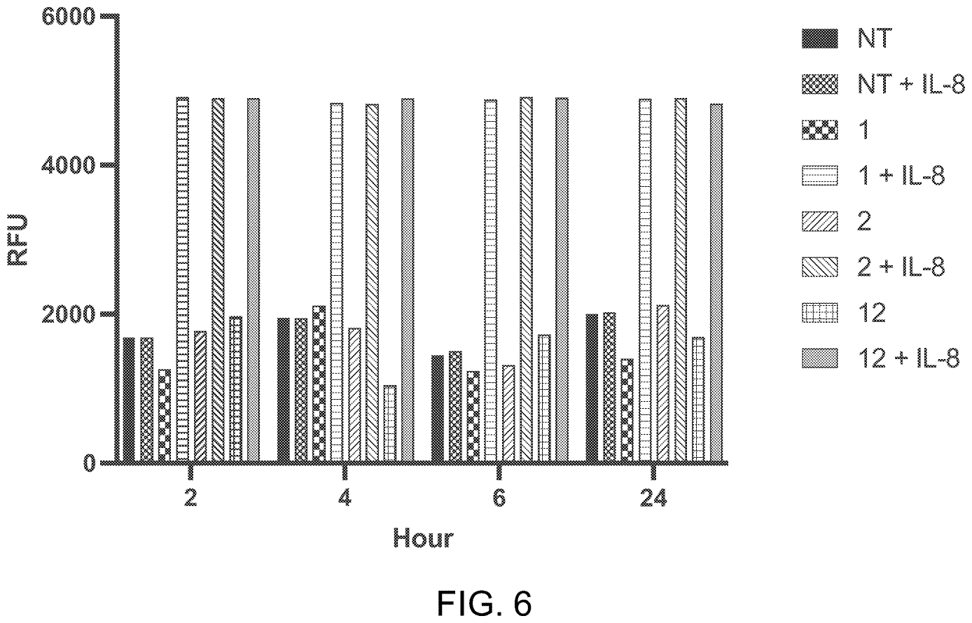

The results demonstrated that transduced IL8 binding protein-transducted T cells (Constructs 1, 2, and 12) had improved migration toward media containing exogenous IL-8 at predetermined concentrations (1,000 pg/mL) compared to media without IL-8, and to non-transduced T cells. This difference is noticed early, and sustained throughout 24 hours. See FIGS. 4A, 4B, and 6.

It is to be understood that the present invention is not to be limited to the exact description and embodiments as illustrated and described herein. To those of ordinary skill in the art, one or more variations and modifications will be understood to be contemplated from the present disclosure. Accordingly, all expedient modifications readily attainable by one of ordinary skill in the art from the disclosure set forth herein, or by routine experimentation therefrom, are deemed to be within the true spirit and scope of the invention.

1. An engineered immune cell engineered to express, on the cell surface, an IL-8-binding protein capable of binding to IL-8.

2. The engineered immune cell of claim 1, wherein the IL-8-binding protein comprises:
(1) a dominant negative IL-8 receptor (DNR) incapable of transducing IL-8 signaling upon IL-8 binding, or
(2) an antigen-binding portion of an antibody specific for IL-8.

3. The engineered immune cell of claim 2, wherein the IL-8-binding protein:
(1) lacks intracellular signaling domain(s) of CXCR1/CXCR2, and/or
(2) transmits an activating signal for the engineered immune cell upon binding IL-8.

4. The engineered immune cell of claim 1, wherein the IL-8-binding protein is encoded by an expression construct (e.g., a vector) introduced into said immune cell.

5. The engineered immune cell of claim 4, wherein the expression construct/vector is a retroviral vector.

6. The engineered immune cell of claim 1, wherein the engineered immune cell is specific for WTl, PRAME, or Survivin.

7. The engineered immune cell of claim 1, wherein the engineered immune cell is a B cell, a natural killer (NK) cell, or a T cell.

8. The engineered immune cell of claim 7, wherein the T cell is a helper T cell, a cytotoxic T cell, a NK T cell, an iNK T cell, a gamma delta T cell, an alpha beta T cell, an antigen-specific T cell, a tumor-infiltrating lymphocyte (TIL), an engineered T cell receptor (TCR) cells, an engineered CAR-T cell, or an antigen-specific T cell.

9. The engineered immune cell of claim 8, wherein the antigen-specific T cell is a tumor-associated antigen-specific T (TAA-T) cell, a neoantigen specific T cell, or a virus specific T cell.

10. A method of treating cancer in a subject in need thereof, the method comprising administering to the subject a therapeutically effective amount of the immune cell of claim 1.

11. The method of claim 10, wherein the cancer is a pediatric solid tumor.

12. The method of claim 11, wherein the pediatric solid tumor is Wilms tumor, rhabdomyosarcoma, neuroblastoma, or melanoma.

13. The method of claim 10, wherein the cancer expresses or over-expresses IL-8.

14. The method of claim 10, wherein the cancer is a chemo-refractory cancer.

15. The method of claim 10, wherein the immune cell is specific for WTl, PRAME, or Survivin.

16. A method of producing the immune cell of claim 1, the method comprising introducing, into an isolated immune cell, an expression construct (e.g., vector) encoding an IL-8-binding protein capable of binding to IL-8, in order to enable expression of the IL-8-binding protein on the surface of the immune cell.

17. The method of claim 16, wherein the expression construct is a lentiviral or retroviral vector.

18. The method of claim 16, wherein the IL-8-binding protein comprises:
(1) a dominant negative IL-8 receptor (DNR) incapable of transducing IL-8 signaling upon IL-8 binding, or
(2) an antigen-binding portion of an antibody specific for IL-8.

19. The method of claim 16, wherein the IL-8-binding protein:
(1) lacks intracellular signaling domain(s) of CXCR1/CXCR2, and/or
(2) transmits an activating signal for the engineered immune cell upon binding IL-8.

20. The method of claim 16, wherein the immune cell is specific for WTl, PRAME, or Survivin.

21-23. (canceled)
